(19) United States
(12) Patent Application Publication
(10) Pub. No.: US 2006/0094106 A1
(43) Pub. Date: May 4, 2006

(54) Biodegradation of subsurface contaminants by injection of gaseous microbial metabolic inducer

(76) Inventors: Lamar E. Priester III, Irmo, SC (US); Brian Harmison, Oak Hill, VA (US); John Huff, Finksburg, MD (US)

Correspondence Address: FOLEY AND LARDNER LLP;SUITE 500, 3000 K STREET NW, WASHINGTON, DC, 20007, US

(21) Appl. No.: 11/225,859

(22) Filed: Sep. 14, 2005

Related U.S. Application Data
(63) Continuation-in-part of application No. 10/394,646, filed on Mar. 24, 2003.
(60) Provisional application No. 60/366,549, filed on Mar. 25, 2002.

(51) Int. Cl. C01B 31/20; C12N 1/38

(52) U.S. Cl. 435/244; 252/372; 435/262.5; 405/128.5

(57) ABSTRACT

The present invention provides a method and a gaseous composition for bioremediation of soil and groundwater contaminated with organic compounds, including halogenated organic compounds and explosives. The gaseous composition contains (a) at least one gaseous microbial metabolic inducer and (b) a carrier gas. The gaseous composition may also optionally include one or more of a gas phase nutrient, a gaseous carbon source, a gas phase reductant, and a moisture source.

### CROSS-REFERENCE TO RELATED PATENT APPLICATIONS

This application is a continuation in part of U.S. patent application Ser. No. 10/394,646, filed Mar. 24, 2003, which claims the benefit of U.S. Provisional Application No. 60/366,459, filed Mar. 25, 2002, both of which are incorporated herein by reference in their entireties.

### BACKGROUND OF THE INVENTION

Chemical contamination of subsurface environments damages local ecosystems and poses health risks where groundwater is used as a source of drinking or irrigation water. Such contamination emanates from various industrial and municipal sources including chemical storage sites, landfills, transportation facilities, and storage tanks located above ground and underground.

A number of methods for treating contaminated soil and groundwater have been available for some time. For example, soil may be excavated, treated at an off-site facility, incinerated and/or disposed. Other methods involve bioremediation techniques. Bioremediation methods employ natural processes to degrade contaminated soil or water. Such methods effectively treat a variety of contaminants. For example, contaminated groundwater may be pumped to the surface and treated to remove or degrade contaminants; similarly, contaminated soil can be removed from a site and treated with biological organisms (Buchanan, U.S. Pat. No. 5,622,864; Stoner et al., U.S. Pat. No. 5,453,375). These methods, however, tend to be expensive and laborious, they require long times for effective treatment, and they carry the risk of exposing contaminants to the atmosphere.

Alternative bioremediation techniques known in the art provide a supply of nutrients in situ via injection wells, thereby circumventing the need to pump or otherwise move contaminated material to the ground surface. These techniques increase bioremediation rates by furnishing heightened concentrations of nutrients to indigenous microbial populations that are capable of degrading contaminants. For example, Looney et al. (U.S. Pat. No. 5,480,549) describe a method by which vapor-phase phosphates such as triethylphosphate and tributylphosphate are metered into a gas stream that is injected via injection wells into contaminated soil and groundwater to stimulate the microbial degradation of hydrocarbon contaminants. The effectiveness of this method, however, is limited to the biodegradation of hydrocarbon contaminants, and is thus inadequate to bioremediate sites where more pernicious contaminants such as halogenated hydrocarbons (halocarbons) persist.

Halocarbons are ubiquitous and are used for a variety of purposes such as dry cleaning agents, degreasers, solvents, and pesticides. Unfortunately, they are one of the most pervasive and harmful classes of contaminants in ground water and soil. Chlorinated hydrocarbons (chlorocarbons) such as tetrachloroethylene (PCE), trichloroethylene (TCE), dichloroethylene (DCE), and vinyl chloride (VC), are exemplary common contaminants. This class of compounds is more resistant to microbial degradation, and thus tends to persist for long periods in the environment. Because the halogen atoms in halocarbons increase the oxidation potential of the carbon atoms to which they are bound, aerobic biodegradation processes are energetically less favorable, particularly for highly halogenated compounds. Consequently, highly halogenated compounds are much more susceptible to anaerobic degradation.

Organic compounds generally act as electron donors. Polyhalogenated compounds, however, behave as electron acceptors in reducing environments as a consequence of the presence of electronegative halogen substituents. Thus, more highly halogenated compounds are less susceptible to aerobic degradation, and more susceptible to anaerobic degradation.

In the environment, halogenated compounds may be naturally dehalogenated by a variety of chemical reactions and microbe-mediated reactions. Some compounds are transformed into products which are more degradable than the parent compounds, or may be more degradable under different environmental conditions. For example, PCE which has been recently released into soil and groundwater will not have degraded much; thus degradation (dehalogenation) will operate on mostly PCE and will be most efficacious in an anaerobic environment. A very old release of PCE, however, will have been naturally dehalogenated to some extent into daughter compounds TCE, DCE, and VC, which are most readily degraded in aerobic environments.

Some environments are inhabited by chemoheterotrophic microorganisms, which may be capable of anaerobically metabolizing existing carbon sources, resulting in the evolution of excess hydrogen (H₂). In the resultant reducing environment, PCE may undergo dehalogenation to TCE. Similarly, TCE may be dehalogenated to DCE and VC. As mentioned above, these latter products are not readily degraded in anaerobic conditions, but can be oxidized under aerobic conditions.

The bioremediation of soil and groundwater contaminated with highly chlorinated hydrocarbons is known in the art. Methods of stimulating the activity of indigenous microbes capable of degrading halocarbons has been achieved by treating subsurface environments with certain carbon nutrients, such as corn syrup and yeast extract (Keasling et al., U.S. Pat. No. 6,150,157) and molasses (Suthersan, U.S. Pat. Nos. 6,143,177 and 6,322,700). These methods, however, require the use of many injection wells, and are limited to the remediation of groundwater where the carbon sources are able to be dispersed. Consequently, they are not practical for the remediation of vadose zones, where the mobility of nutrients such as corn syrup and molasses is negligible. One attempt to overcome these limitations was disclosed by Hughes et al., (U.S. Pat. No. 5,602,296), whose method entails the injection of pure hydrogen (H₂) into contaminated subsurface regions. Reductive dechlorination of chlorinated hydrocarbons was suggested to be mediated by indigenous anaerobic bacteria. This method, however, creates a strongly reducing environment and is thus ineffective for the degradation of partially chlorinated hydrocarbons such as DCE and VC. Moreover, it is ineffective in the treatment of nonhalogenated contaminants. Finally, hydrogen is extremely flammable, and thus poses a serious health risk where it is used as a pure gas.

Perchlorate contamination is becoming a more widespread concern in the United States as sources of such contamination continue to be identified and as more sensitive analytical methods are developed that can detect this compound in soil and groundwater. Perchlorate contamination is of particular concern because of the persistent and toxic nature of this chemical and because its physical and chemical properties make it challenging to treat. In addition to its use as an oxidizer in propellants and explosives, perchlorate has a wide variety of uses in areas ranging from electronics manufacturing to pharmaceuticals.

In situ bioremediation (ISB) is a technology used frequently to treat perchlorate in contaminated groundwater and soil. It uses microorganisms capable of reducing perchlorate to chloride and oxygen under anaerobic conditions. This process requires supply of electron donor and an appropriate substrate to support microbial growth. ISB has reduced perchlorate concentrations to less than 4 ug/L in groundwater.

In situ bioremediation (ISB) is a controlled biological process in which microorganisms convert perchlorate to chloride and oxygen. Bioremediation reduces perchlorate via enzymatic degradation by select species of bacteria under anaerobic conditions. This requires an adequate supply of nutrients to support microbial growth (Urbansky and Schock, 1999; Rosen, 2003). According to Urbansky and Schock, “Issues in managing the risks associated with perchlorate in drinking water,” 56 J. Environmental Management 79 (1999), certain bacteria have a natural tendency to degrade perchlorate into chloride and oxygen under anaerobic conditions. These bacteria include: Ideonella dechloratans, Proteobacteria, Vibrio dechloraticans, Cuzensove B-1168, and Wolinella succinogenes HAP-1 (Urbansky and Schock, 1999). Other bacteria capable of reducing perchlorate have been identified in the genera Dechloromonas and Dechlorosoma. See Interstate Technology Regulatory Council (ITRC), “Overview: Perchlorate Overview”, March, 2005; Coates et al., “The ubiquity and diversity of dissimilatory (per)chlorate-reducing bacteria”, 65 Applied and Environmental Microbiolgy 5234 (1999); Coates et al., “The diverse microbiology of (per)chlorate reduction” in Perchlorate in the Environment (E. Urbansky, ed.), Kluwer Academic/Plenum Publishers, NY (2000), pp 257-270.

ISB of perchlorate typically involves enhancement techniques. Biological degradation of perchlorate requires select species of microorganisms, mostly bacteria, and sufficient amounts of amendments in the form of nutrients and electron donors (Urbansky and Schock, 1999; and Owsianiak et al., “In Situ Removal of Perchlorate from Perched Groundwater by Inducing Enhanced Anaerobic Conditions”, presented at the Seventh International In Situ and On-Site Bioremediation Symposium, Jun. 2-5, 2003). Some commonly used electron donors include organic acids such as acetate, citrate, and lactate; sugars such as glucose; alcohols such as ethanol; and protein-rich substances such as casamino acids and whey (ITRC, 2005). For enhanced ISB, the electron donor and nutrient material are injected into the contaminated zone. Number and spacing of injection points depend on several factors including extent of contaminant plume, design of the injection field (e.g., re-circulation, barrier, or grid), subsurface lithology, and type of material injected. The injected substances cause the perchlorate-reductive reactions to occur within the contaminated media (Owsianiak et al., 2003; Koenigsberg and Willett, “Enhanced In Situ Bioremediation of Perchlorate in Groundwater with Hydrogen Release Compound (HRC©), presented at NGWA Conference on MTBA and Perchlorate, Jun. 4, 2004).

Reactions leading to biological degradation of perchlorate by in situ bioremediation are under investigation. Ongoing research indicates that perchlorate is reduced in a three-step process. First, perchlorate ion is reduced to ClO₃, then to ClO₂, and subsequently to Cl, and O₂. The reactions discussed above are catalyzed by the enzymes perchlorate reductase and chlorite dismutase. See Beisel et al., “Ex Situ Treatment of Perchlorate Contaminated Groundwater”, presented at NGWA Conference on MTBA and Perchlorate, Jun. 4, 2004; Naval Facilities Engineering Command (NAVFAC), available at http://www.perchlorateinfo.com/perchlorate-case-40.html (2000); Polk et al., “Case Study of Ex-Situ Biological Treatment of Perchlorate-Contaminated Groundwater”, presented at the 4^{th} Tri-Services Environmental Technology Symposium, Jun. 18-20, 2001.

Based on the foregoing considerations, there remains a need in the art for a method of biodegradation that is useful against a wide variety of contaminants, including halocarbons, perchlorate compounds and non-halogenated compounds.

### SUMMARY

The present invention relates to the bioremediation of soil and groundwater at sites that are contaminated with various organic substances.

One embodiment of the invention is a gaseous composition comprising (a) at least one gaseous microbial metabolic inducer and (b) a carrier gas.

Another embodiment of the invention is a method of stimulating microbial degradation of at least one pollutant in a subsurface environment, said method comprising contacting the subsurface environment with (a) a gaseous composition comprising at least one gaseous microbial metabolic inducer and (b) a carrier gas.

Another object of this invention is a method of stimulating the in situ microbial degradation of one or more pollutants in a subsurface environment by contacting the subsurface environment with a gaseous, microbially nutritive composition. The composition comprises hydrogen (H₂) and one or more volatile phosphate nutrients, and is introduced to the subsurface environment at a rate, pressure, and time sufficient to degrade said one or more pollutants. The gaseous composition stimulates the growth and reproduction of indigenous bacteria that are capable of degrading the pollutants.

It is another object of the present invention to provide a gaseous, microbially nutritive composition that comprises hydrogen (H₂) and one or more volatile phosphate nutrients. The phosphate nutrient, which may be a liquid under standard conditions, is sufficiently volatile such that a carrier gas containing the hydrogen may readily entrain the phosphate nutrient in its gas phase. Thus, hydrogen and phosphate are delivered to a remediation site in vapor form, and are thereby dispersed effectively throughout the site.

Another aspect of the invention provides a method of stimulating in situ microbial degradation of one or more pollutants in a subsurface environment comprising the step of contacting the subsurface environment with a gaseous, microbially nutritive composition comprising hydrogen (H₂) and one or more volatile phosphate nutrients; wherein the composition is introduced to the subsurface environment at a rate, pressure, and time sufficient to degrade one or more pollutants. The volatile phosphate nutrients may be triethylphosphate (TEP) and tributylphosphate (TBP) in a concentration of 0.001% -1% (v/v); 0.005% -0.5% (v/v); 0.008% -0.02% (v/v); or 0.01% (v/v). The gaseous, microbially nutritive composition may futher comprise 0.01% -10% (v/v); 0.015% -5% (v/v); or 0.1% (v/v) nitrous oxide (N₂O). The composition may further comprise 1% -50%, 1% -10% (v/v) H₂, 2% -7% (v/v) H₂, 3% -5% (v/v) H₂, or 4% (v/v) H₂. Additionally composition may further comprise 0.01% -10% (v/v); 0.015% -5% (v/v); or 0.1% (v/v) nitrous oxide (N₂O). The gaseous, microbially nutritive composition may still further comprise 0.1% -20% (v/v); 2% -6% (v/v); 4% (v/v) carbon dioxide (CO₂). The composition may still even further comprise a volatile alkane such as methane, ethane, propane, butane or pentane. The gaseous, microbially nutritive composition may further comprise a carrier gas such as air, nitrogen (N₂) or a noble gas such as helium (He), neon (Ne) or argon (Ar). The gaseous, microbially nutritive composition may comprise 4% (v/v) H₂; 0.1% (v/v) N₂O; and 0.01% (v/v) TEP, TBP.

In another aspect of the invention there is provided a method of stimulating in situ microbial degradation of organic pollutants in a subsurface environment comprising the step of contacting the subsurface environment with a gaseous, microbially nutritive composition comprising hydrogen (H₂), nitrous oxide (N₂O), one or both of triethylphosphate (TEP) and tributylphosphate (TBP), a carrier gas, and, optionally, a volatile alkane; wherein the composition is introduced to said subsurface environment at a rate, pressure, and time sufficient to degrade said one or more pollutants. The volatile phosphate nutrients may be triethylphosphate (TEP) and tributylphosphate (TBP) in a concentration of 0.001% -1% (v/v); 0.005% -0.5% (v/v); 0.008% -0.02% (v/v); or 0.01% (v/v). The gaseous, microbially nutritive composition may further comprise 0.01% -10% (v/v); 0.015% -5% (v/v); or 0.1% (v/v) nitrous oxide (N₂O). The gaseous, microbially nutritive composition may further comprise 1% -50%, 1% -10% (v/v) H₂, 2% -7% (v/v) H₂, 3% -5% (v/v) H₂, or 4% (v/v) H₂. The composition may further comprise 0.01% -10% (v/v); 0.015% -5% (v/v); or 0.1% (v/v) nitrous oxide (N₂O). The gaseous, microbially nutritive composition may still further comprise 0.1% -20% (v/v); 2% -6% (v/v); 4% (v/v) carbon dioxide (CO₂). The gaseous, microbially nutritive composition may further comprise a volatile alkane such as methane, ethane, propane, butane or pentane. Additionally, the composition may further comprise a carrier gas such as air, nitrogen (N₂) or a noble gas such as helium (He), neon (Ne) or argon (Ar). Finally, the gaseous, microbially nutritive composition may comprise 4% (v/v) H₂; 0.1% (v/v) N₂O; and 0.01% (v/v) TEP, TBP.

The methods of the instant invention are used for biodegradation of pollutants that are optionally substituted unsaturated hydrocarbons, optionally substituted partially saturated hydrocarbons, optionally substituted saturated hydrocarbons, halocarbons, or mixtures thereof. The pollutants may be chlorinated hydrocarbons, monocyclic aromatic hydrocarbons, or polycyclic aromatic hydrocarbons. The pollutants may also be benzene, ethylbenzene, nitrobenzene, chlorobenzene, dinitrobenzenes, toluene, xylenes, biphenyl, halobiphenyls, polyhalogenatedbiphenyls, mesitylene, phenol, cresols, aniline, naphthalene, halonaphthalenes, anthracene, phenanthrene, fluorene, benzopyrenes, styrene, dimethylphenol, halotoluenes, benzoanthenes, dibenzofuran, chrysene, catechol, toluic acids, ethylene dibromide, chloroform, tetrachloroethylene, trichloroethylene, dichloroethylene, vinyl chloride, methyl-tert-butyl ether, hexadecane, methanol, and mixtures thereof.

One advantage of the present invention over conventional remediation techniques is that it does not require the removal of soil or groundwater for treatment and subsequent disposal. Instead, the biodegradation of pollutants occurs entirely in situ within a subsurface region. Thus, the present invention presents very little risk of pollutants being released into the atmosphere.

Another advantage afforded by the present invention is that it is straightforward to implement. The equipment is simple and the materials employed are readily obtained. Additionally, remediation occurs during much shorter time frames than with traditional remediation technologies.

Other features and advantages of the present invention will become apparent to those skilled in the art from a careful reading of the Detailed Description presented below.

### DETAILED DESCRIPTION

As described in detail herein, the present invention provides a highly efficient, unique method for biodegrading a wide variety of pollutants.

In one embodiment, the volatile phosphate nutrient entrained in a carrier gas is a mixture of triethylphosphate (TEP) and tributylphosphate (TBP). Alternatively, TEP or TBP may be used as the sole phosphate source. Both TEP and TBP exhibit high vapor pressures, and thus mix easily with a carrier gas so that a high concentration of the nutrient may be delivered throughout a bioremediation site. Additionally, TEP and TBP are relatively benign and are the safest phosphate compounds which can be vaporized.

In another embodiment, the composition comprises a carrier gas. The carrier gas can be selected to facilitate either aerobic or anaerobic environments. Where an aerobic environment is desired, the carrier gas comprises air. Where an anaerobic environment is desired, the carrier gas is inert. An illustrative gas is nitrogen (N₂). Alternatively, the inert carrier gas can contain a noble gas. Other specific examples of noble gases are helium, neon, and argon. Thus, the skilled artisan will determine whether biodegradation is most efficacious in an aerobic or anaerobic environment, and can readily adjust the carrier gas accordingly.

In addition to hydrogen (H₂) and a volatile phosphate, the gaseous composition of the present invention optionally contains other components. In one embodiment, the composition contains nitrous oxide (N₂O), which serves as an additional nutrient to indigenous microbes at the remediation site. In most circumstances, injection of a gaseous composition containing the volatile phosphate nutrient and hydrogen is sufficient to effectively bioremediate a polluted site.

In another embodiment of the present invention, the gaseous composition contains a volatile alkane. An alkane is a fully saturated hydrocarbon that can serve as an additional microbial energy source where especially pernicious contaminants such as halocarbons are present. Examples of a volatile alkane include methane, ethane, propane, butane, and pentane. Finally, the gaseous composition may comprise carbon dioxide (CO₂), which can lower the pH of a particularly alkaline subsurface region. An exemplary composition in this regard comprises hydrogen, nitrous oxide, one or both of TEP and TBP, a carrier gas, and an optional volatile alkane.

As mentioned above, the present invention is useful in the biodegradation of numerous pollutants. The pollutants can be organic compounds, such as those typically associated with petroleum waste products. For example, these include optionally substituted unsaturated hydrocarbons, optionally substituted partially saturated hydrocarbons, optionally substituted saturated hydrocarbons, halocarbons, or mixtures thereof. More specifically, the pollutants are chlorinated hydrocarbons, monocyclic aromatic hydrocarbons, and polycyclic aromatic hydrocarbons. Examples of these classes of pollutants include, but are not limited to benzene, ethylbenzene, nitrobenzene, chlorobenzene, dinitrobenzenes, toluene, xylenes, biphenyl, halobiphenyls, polyhalogenatedbiphenyls, mesitylene, phenol, cresols, aniline, naphthalene, halonaphthalenes, anthracene, phenanthrene, fluorene, benzopyrenes, styrene, dimethylphenol, halotoluenes, benzoanthenes, dibenzofuran, chrysene, catechol, toluic acids, ethylene dibromide, chloroform, tetrachloroethylene, trichloroethylene, dichloroethylene, vinyl chloride, methyl-tert-butyl ether, hexadecane, methanol, and mixtures thereof.

A major advantage of the present invention is that its practical application employs inexpensive and readily available equipment such as standard blowers, nitrous oxide tanks, piping, valves, and pressure gauges. For example, the individual gaseous components of the present invention are readily available from commercial sources and are conveniently stored in and dispensed from routine containers employed in the art, including but not limited to cylinders or dewars, bulk transfer tanks, and cryogenic storage tanks. Additionally, some of the components such as hydrogen can be generated through on-site generation, employing means such as sieves, membranes, electrolysis, or fuel cell production.

While not complex, this equipment is utilized at remediation sites where the subsurface environment is typically characterized by heterogeneous physical, chemical, and biological compositions. To ensure that pollutants at a remediation site are effectively eliminated in such an environment, the present invention provides a high degree of control over operating parameters such as the depth, volume, and pressure with which the gaseous composition is injected. Thus, variations in soil properties and stratigraphy may be compensated for by judicious control of these parameters. Additionally, naturally occurring organisms present in subsurface regions may compete for hydrogen. Consequently, those skilled in the art can judiciously correct the concentration of injected hydrogen, taking into account this additional consumption of hydrogen on a site-by-site basis. Other soil properties that may affect the transmission of pollutants and vapors through the subsurface environment can be determined by soil bore surveying techniques that are known to those who are skilled in the art. For example, such techniques are described by Johnson, et al., in “A Practical Approach to the Design, Operation, and Monitoring of In Situ Soil-Venting Systems” in Ground Water Monitoring Review 10, no. 1, 1990, pp. 159-178, and by G. D. Sayles in “Test Plan and Technical Protocol for a Field Treatability Test for Bioventing” from the Environmental Services Office, US Air Force Centers for Environmenal Excellence (AFCEE), May 1992.

The gaseous composition may be introduced to a subsurface environment through one or more injection points, the number of which needed may be readily determined by a person skilled in the art. Because the present invention utilizes a gaseous nutritive composition, in contrast to prior art methods using liquid compositions, the injection points may be situated such that the gaseous composition is either sparged into groundwater in the saturated zone, biovented into the vadose zone, or both. Flow rates of the gaseous composition can range from 0.5 to 7 cubic feet per minute (CFM) per injection point. The pressure at which the gaseous composition is injected varies widely, and must be determined on a site-by-site basis. Generally, the injection pressure depends upon factors including the depth at which the gaseous composition is injected and whether it is injected above, in, or below ground water.

The nutrients conveyed to a subsurface environment by the method of the present invention, together with nutrients that are already present at a remediation site, optimize the growth of pollutant-degrading microbes and the rate at which pollutants are degraded. Microbes utilize carbon, nitrogen, and phosphorus in approximately the same ratios as their own bulk C:N:P ratio. Optimum stimulation of a microbial population can be achieved when the gaseous composition of the present invention is tailored to match this C:N:P ratio, which may differ depending not only on the kind of microbe, but on environmental conditions such as the types of pollutants, availability of water, soil pH, and oxidation-reduction potentials. Thus, the optimum C:N:P ratio of the gaseous composition is specific to the conditions of a given remediation site.

The amount of volatile phosphate contained in the gaseous composition varies. In a typical application of the present invention, the concentration of volatile phosphate ranges from 0.001% -1%. In some embodiments the concentration ranges from about 0.005% -0.5% or from about 0.008-0.02% (v/v). An exemplary amount of volatile phosphate is 0.01% (v/v).

The concentration of hydrogen can also vary and must be adjusted according to the particular needs at a remediation site. Hydrogen is consumed in the microbe-mediated reductive dehalogenation of halogenated pollutants, particularly those with high halogen content. It is theoretically possible to use high concentrations of hydrogen, such as those used in the prior art. However, practical considerations such as electrical conduits and other potential sources of ignition present in urban areas where subsurface contamination normally arises will limit the concentration of hydrogen to safe levels. Typically, the concentration of hydrogen in the gaseous composition can vary from about 1% -50%, about 1% -10%, about 2% -7%, and about 3-5% (v/v). An exemplary amount of hydrogen is about 4% (v/v).

In some subsurface regions, the amount of naturally occurring nitrogen needed to support microbial growth may be unsuitably low. Therefore, the gaseous composition of this invention may need to be supplemented with nitrous oxide (N₂O). When nitrous oxide is used, it is typically present in the amount of about 0.01% -10%, or about 0.015% -5%. An exemplary amount of nitrous oxide is about 0.1% (v/v).

The gaseous composition can contain other components. As mentioned above, a volatile alkane may be used as an additional microbe nutrient. Typically, the alkane is present in the amount of 1% -10% (v/v). Carbon dioxide may be used to lower the pH of particularly alkaline environments. When it is used, carbon dioxide is present in the gaseous composition in the amount of about 0.1% -20% or about 2% -6% (v/v). An exemplary amount of carbon dioxide is about 4% (v/v).

The method of the present invention is applicable to sites contaminated with a wide variety of contaminants. The concentration of contaminants that remain at a site after treatment by the gaseous composition of this invention can be reduced to levels below detectable limits.

Another embodiment of invention is a gaseous composition comprising at least one gaseous microbial metabolic inducer. The gaseous inducer can be, for example, an inducer of alkB and/or alkS expression. The inducers of alkB and/or alkS expression are generally known in the art. See, e.g. Smits et. al. 2001, Plasmid 46(1):16-24; Grand, A., et. al. Journal of Bacteriology, 1975, vol. 123(2); p. 546-556; Eggink, G., et. al. Journal of Biological Chemistry, 1988, vol. 263(26), p. 13400-13405; Panke, S. et. al., 1999, vol. 65(6), p. 2324-2332, which are all incorporated herein by reference in their entireties. The gaseous inducer can be, for example, n-alkanes having 6 to 12 carbon atoms, alkenes, haloalkanes, volatile acetates such as ethyl acetate, volatile ethers such as diethyl ether, dicyclopropylketone (DCPK), dicyclopropylmethanol (DCPM), isopropyl-β-D-thiogalactopyranoside (IPTG) or any combination thereof. Combinations of two or more gaseous inducers are also contemplated.

In one embodiment, the gaseous inducer comprises DCPK. The concentration of DCPK in the gaseous composition can range, for example, from about 0.0005% to about 10% volume to volume (v/v), from about 0.001% to about 5% (v/v), or from about 0.01% to about 1% (v/v). An exemplary amount of DCPK in the composition can be about 0.05% (v/v).

The gaseous composition can further comprise a carrier gas. In some embodiments, the carrier gas can be air. In other embodiments, the carrier gas can be chemically inert gas such as nitrogen, helium, neon, argon or any combination thereof. The carrier gas can be selected to facilitate either aerobic or anaerobic environments. Where an aerobic environment is desired, the carrier gas comprises air. Where an anaerobic environment is desired, the carrier gas is inert. An illustrative inert gas is nitrogen (N₂). Alternatively, the inert carrier gas can contain a noble gas. Other examples of noble gases are helium, neon, and argon. Thus, the skilled artisan will determine whether biodegradation is most efficacious in an aerobic or anaerobic environment, and can readily adjust the carrier gas accordingly.

In some embodiments, the gaseous composition can further comprise a gaseous reductant such as hydrogen (H₂). Typically, the concentration of hydrogen in the gaseous composition can vary from about 1%—about 50%, about 1%—about 10%, about 2%—about 7%, and about 3—about 5% (v/v). An exemplary amount of hydrogen can be about 4% (v/v).

The gaseous composition can further comprise at least one gas phase nutrient. The gas phase nutrient can comprise, for example, a volatile phosphate such as trimethylphosphate, triethylphosphate, tripropylphosphate, tributylphosphate or any combination thereof The amount of volatile phosphate in the gaseous composition can vary. In a typical application of the present invention, the concentration of volatile phosphate can range from about 0.001%—about 1%, about 0.005%—about 0.5%, and about 0.01—about 0.1% (v/v). An exemplary amount of volatile phosphate can be about 0.05% (v/v). In some embodiments, the gas phase nutrient can comprise nitrous oxide (N₂O). When nitrous oxide is used, it can be typically present in the amount of about 0.01%—about 10% or about 0.015%—about 5%. An exemplary amount of nitrous oxide can be about 0.1% (v/v).

The gaseous composition can further comprise at least one gaseous carbon source. The gaseous carbon source can comprise a volatile/gaseous alkane, a volatile alcohol, a volatile ether or any combination thereof. The volatile alkane can be, for example, a methane, ethane, propane, butane (including isomers), pentane (including isomers), hexane (including isomers), heptane (including isomers), octane (including isomers), nonane (including isomers), decane (including isomers) or any combination thereof. The volatile alcohol can be, for example, methanol, ethanol, 1-propanol, 2-propanol, 1-butanol, 2-butanol or any combination thereof. The volatile ester can be, for example, ethyl acetate.

In some embodiments, the gaseous composition can further comprise a moisture source such as steam or humidified air. The gaseous composition comprising the moisture source can be particularly useful in arid environment. The amount of moisture in the gaseous composition can range from about 0.0001% to about 5%.

The gaseous composition can be used for stimulating microbial biodegradation of at least one pollutant or contaminant in a subsurface environment such as soil/subsoil or a groundwater by contacting the gaseous composition with the subsurface environment. The stimulating of microbial degradation by the gaseous composition can be performed in-situ or ex-situ. In one embodiment, the method is performed in-situ, meaning that the gaseous composition is applied to the subsurface environment at its natural location. In another embodiment, the method is performed ex-situ, meaning that contaminated soil, water, or both, for example, can be removed from its natural location to be treated by the gaseous composition.

The gaseous composition can be introduced to the subsurface environment at a rate, pressure, and time sufficient to degrade said one or more pollutants. In some embodiments, contacting the gaseous composition with the subsurface environment can be carried out continuously. Yet in some embodiments, contacting the gaseous composition with the subsurface environment can be performed in pulses. The duration and the frequency of pulses can be determined by one skilled in the art. Specific duration and pulsing can be determined by the skilled person in light of, for example, the lithology, groundwater and soil chemistries, and target contaminant(s).

In some embodiments, contacting the composition with the subsurface environment can comprise injecting the gaseous composition at a first point and then extracting the composition at a second point, thus pulling the composition through a contaminated area of the subsurface environment. The injection and the extraction can be carried out through wells, such as injection or monitoring wells, or through piping such as perforated piping. The extraction of the composition can involve applying negative pressure at the second point.

The gaseous compositions of the present invention can be applied for stimulating of microbial degradation of the variety of pollutants including but not limited to 1) alkanes; 2) alkenes; 3) chlorinated alkanes, including, but not limited to trichloroethane (TCA) and dichloroethane (DCA); 4) chlorinated alkenes, including, but not limited to tetrachloroethene (PCE), trichloroethylene (TCE), dichloroethylene (DCE) and vinyl chloride (VC); 5) aromatic compounds, including, but not limited to benzene, toluene, ethylbenzene, xylene, and styrene; 6) explosive compounds, including, but not limited to cyclotetramethylenetetranitramine (HMX), cyclotrimethylenetrinitramine (RDX) and trinitrotoluene (TNT); 7) pesticides; 8) polychlorinated biphenyls; 9) Dowtherms® and Dowtherm components including but not limited to phenyl benzene, phenoxybenzene, ethylene glycol, propylene glycol and 10) perchlorate compounds including perchlorate ions in solutions.

When at least one of the pollutants is a perchlorate compound, a typical gaseous composition contacted with a subsurface environment can comprise (a) dicyclopropylketone as said gaseous inducer, (b) nitrogen as said carrier gas, (c) triethylphosphate, trimethylphospate or a combination thereof, (d) hydrogen (H₂), (e) propane, (f) optionally, ethanol, and, (g) optionally, ethylacetate. In some embodiments, the gaseous composition can comprise both ethanol and ethylacetate. In the above composition, the concentration the phosphate nutrient can range from about 0.001 to about 10% (v/v), from about 0.01 to about 1%, or from about 0.02 to about 0.1% (v/v). An exemplary amount is about 0.05% (v/v). The concentration of DCPK can range from about 0.001% to about 10%, from about 0.01% to about 2%; or from about 0.05% to about 0.5%. An exemplary concentration is about 0.2% (v/v). The concentration of ethylacetate can range from about 0% to about 50%, from about 1% to about 20%, or from about 8 to about 12% (v/v). The concentration of ethanol can range from around 0 to about 30%, from about 1 to about 15%, or from about 5% to about 10%.

The following examples are provided to further describe the invention by way of specific embodiments of the invention. The examples are intended to be non-limiting illustrations of the invention.

### EXAMPLE 1

A contaminant plume containing highly chlorinated compounds such as methylene chloride, and TCE located in Herlong, Calif. was subjected to an injection regimen initially designed to induce an anaerobic, reducing environment that is rich in hydrogen and carbon, and containing sufficient nitrogen and phosphorus to support rapid cell growth of indigenous microbes. The gaseous microbially nutritive composition comprised nitrogen as the carrier gas at a concentration of 50% together with a hydrogen source at a concentration of 45%, a propane at a concentration of 4%, nitrous oxide at a concentration of 0.1% and vapor phase TEP at a concentration of 0.01%. TEP was introduced into the gaseous composition by passing the composition (less TEP) through a cylinder gas manifold with rotameters and mixing tubes and contacting it with TEP in a head space contactor. The gasoues microbially nutritive composition was injected into a subsurface region for 8 hours per week for 8 weeks through a sparge point located 100 feet below ground surface. As discussed above, hydrogen provided for the immediate dechlorination of methylene chloride and TCE. Eventually, the growing biomass naturally supplemented the hydrogen supply.

Prior to the injection of the gaseous microbially nutritive composition the concentration of methylene chloride in the groundwater was 117 ppb as determined by the EPA Method 8260. Following the first two weeks of treatment the methylene chloride concentration was reduced to less than detection limits (<1 ppb).

### EXAMPLE 2

### Gas Phase Enhancements for Perchlorate Bioremediation—Bench Scale Microbial Reduction Study

Several microcosm systems were treated according to the invention as generally described above. Several soil and groundwater reaction vessels containing soil and contaminated groundwater were obtained from Mortandad Canyon on Los Alamos National Laboratory (LANL) property. An area in upper Mortandad Canyon where contaminants have migrated to 50-200 feet below ground surface was chosen for the study. Soil cuttings were obtained from a perched area during a new well construction. The cuttings were composited for this study, and packaged in five, 5 gallon containers. Groundwater was obtained from a nearby monitoring well. Treatment constituents and regimen were intended to induce an anaerobic micro-environment conducive to reductive metabolic mechanisms. The purpose of the microcosm study was to demonstrate that gas phase carbon sources and metabolic inducers are capable of sustaining an environment supporting microbial reduction of perchlorate to chloride ion and oxygen in-situ, and to determine the lower concentration limit of reduction of perchlorate by the method.

The microcosm system was designed to allow timed and metered injection of treatment constituents individually, in order to approximate processes and treatment regimen that may be employed during a field pilot scale project. The system is flexible and allows modification of treatment constituents and treatment regimen in real time in response to study results.

The soil/groundwater was contained in new food or laboratory grade plastic containers, which were decontaminated (Alconox wash, isopropyl alcohol rinse, distilled water rinse) prior to filling with test materials. Containers were calibrated to facilitate filling with approximately 75% soil/rock and 25% groundwater test material per container. This required approximately 3-4 gallons of soil and 0.20 gallons of groundwater per container. Five containers were utilized, requiring approximately 20 gallons of soil and 1 gallon of contaminated groundwater for the study.

Test materials consisted of soil and contaminated groundwater obtained from the Mortandad Canyon site. Each test container was filled with a mixture of soil/rock matrix and groundwater of known concentration of perchlorate. Soil and groundwater samples obtained during test material collection were analyzed by a certified laboratory and Method 314 (U.S. EPA, method 314.0, revision 1.0 (1999), Determination of Perchlorate in Drinking Water Using Ion Chromatography with Mass Spectrometry) to ensure that appropriate perchlorate concentrations were utilized for the study. The initial sampling indicated that the soil contained no detectable perchlorate and the groundwater concentrations were very low (20-20.7 μg/l).

It was decided to spike the groundwater with sodium perchlorate and use the spiked groundwater to add perchlorate to the soil microcosms.

Five reaction containers were prepared:
#1 consisted of soil plus groundwater and was not treated with any constituents to act as a control;
#2 consisted of soil plus groundwater and was treated with the base injection mixture;
#3 consisted of soil plus groundwater and was treated with the base injection mixture plus ethyl acetate;
#4 consisted of soil plus groundwater and was treated with the base injection mixture plus ethanol;
#5 consisted of soil plus groundwater and was treated with the base injection mixture plus a proprietary mixture of volatile carbon sources and a metabolic inducer (dicyclopropylketone).

Each reaction container was constructed with a false bottom to retain the soil approximately ¾ inches from the container bottom. One injection point was installed in the container floor to allow saturation of the horizontal and vertical aspect of the soil column. The containers were tightly sealed and outfitted with an airlock to maintain a sustained anaerobic environment.

The injection system comprised a nitrogen gas source (cylinder) capable of delivering a minimum of 0.1 cubic feet per minute (CFM) at approximately 2 PSI to each treated container; a hydrogen gas source (cylinder) capable of delivering approximately 3% (v/v) hydrogen to the nitrogen stream; a propane gas source (cylinder) capable of delivering approximately 1% (v/v) propane to the nitrogen stream; and a liquid contactor capable of supplying approximately 0.04% (v/v) volatilized triethylphosphate (TEP)/trimethylphosphate (TMP) gas mixture to the nitrogen stream and a similar contactor was used for the proprietary mixture and DCPK. The auxiliary carbon source contactors were designed based on Dalton's Law to provide the appropriate gas phase carbon and DCPK concentrations, in the base carrier gas mixture.

The injection system was controlled by compressed gas regulators, manual timers, and valves to allow the controlled injection of each injection constituent as dictated by the injection regimen.

The injection system included in-line flow meters to allow control and adjustment of the flow rate and absolute concentration of nitrogen, propane and hydrogen injection constituent.

Although the reaction containers were not stored in a refrigerated environment, the environmental temperature was monitored and documented throughout the study period. The temperature in the laboratory area was maintained between 65 and 70 degrees F.

Treatment Protocol

The injection protocol included the following treatment constituents and metered quantities (for reaction containers #002, #003, #004 and #005):
Reaction Container #002 received the “base mixture” containing
   Approximately 6 cubic feet per hour (CFH) nitrogen
   Approximately 0.18 CFH hydrogen
   Approximately 0.06 CFH propane
   Approximately 0.03% volatilized TEP/TMP
Reaction Container #003 received the base mixture plus injection of gas phase ethyl acetate
Reaction Container #004 received the base mixture plus injection of gas phase ethanol
Reaction Container #005 received the base mixture plus a gas phase blend of carbon sources such as propane, ethyl acetate, and/or ethanol, together with gas phase DCPK.

Reaction containers #002, #003, #004 and #005 were initially treated with a 90 minute injection of the above constituents followed by a second 90 minute injection after 30 days.

Reaction container #001 received no treatment, but soil and groundwater was sampled and analyzed on the same schedule as the other three reaction containers.

Sampling and Analysis

The parameters listed in Table 1 were tested in accordance with the prescribed analytical method:

TABLE 1
Test Parameters and Analytical Methods, Groundwater

| Parameter | Method |
|---|---|
| Nitrate-Nitrite | 353.2* |
| Perchlorate-chlorate-chlorite | 314.0* |
| Sulfate as SO4 | 375.4* |
| Chloride | 4500E |
| Ferrous Iron (2+) | SM3500 |

*U.S. EPA drinking water analytical methods.

Soil samples were collected at the time zero sampling event and sent to a qualified laboratory for enumeration of perchlorate-reducing microorganisms, and analysis by DNA probe for chlorite dismutase gene cld. Soil samples and groundwater samples were submitted to an environmental laboratory for analysis of perchlorate and reduction products by EPA Method 314.0. Perchlorate in the soil samples was extracted using standard methods.

After the time zero sampling, additional soil samples were withdrawn from each reaction container and sent for analysis four weeks after treatment. This analysis will include the parameters presented in Table 1 above. Soil samples were sent for additional microbiological analysis at the treatment conclusion. Samples were composited from within the container for this final analysis. One sample from each container was analyzed.

A laboratory certified by the EPA to perform environmental analytical testing (Severn Trent Laboratories, Inc. in Savannah, Ga.) performed the perchlorate, chlorate and chlorite analyses through Access Analytical, Inc. of Irmo, S.C. The laboratory provides a 10-14 day standard turnaround time for perchlorate samples. Microbiological testing (MPN enumeration) and chlorite dismutase gene cld enzyme assay were performed by Biolnsite, Inc of Carbondale, Ill.

At the time of sample collection, “field analysis” of water samples was performed which included dissolved oxygen, pH, specific conductivity, and oxidation/reduction potential (ORP) for each sample.

### RESULTS AND CONCLUSIONS

Background

It is widely accepted that perchlorate is used as an electron acceptor by some bacteria for cellular respiration and is degraded completely to chloride ion. The bacteria that degrade perchlorate are diverse. Almost all of them fall within classifications based on a 16s rDNA classification scheme—a recombinant DNA methodology based on the 16s gene, which can be used to assess the phylogeny of bacteria. Most perchlorate-respiring microorganisms (PRMs) are capable of functioning under varying environmental conditions and use oxygen, nitrate, and chlorate (ClO₃)—but not sulfate—as a terminal electron acceptor. Perchlorate can be successively degraded to chlorate and then chlorite (ClO₂⁻) by a novel chlorate reductase respiratory enzyme. A chlorate-respiring bacterium was the first isolate shown to be capable of benzene degradation, although only under denitrifying, and not chlorate-reducing, conditions.

Because chlorite is toxic to bacteria, the key to bacterial growth using chlorate and perchlorate is the presence of chlorite dismutase, a nonrespiratory enzyme that catalyzes the disproportionation of chlorite to O₂ and Cl⁻. Rates of chlorite disproportionation by chlorite dismutase are greater than chlorate reduction by chlorate reductase and oxygen utilization by cytochromes; the slowest step is perchlorate reduction. As a result, no intermediates ordinarily accumulate in solution during perchlorate biodegradation. In fact, the heme-based chlorite dismutase is produced in such large quantities by PRMs that the addition of chlorite to a concentrated cell suspension grown anaerobically on chlorate or perchlorate will produce visible frothing due to O₂ release.

Results

The microcosms were set up in November 2004, after receipt of the soil. The five microcosms were sampled Nov. 23, 2005 and subsequently determined to contain non-detectable levels of perchlorate. Three one hour weekly injections had been performed to the microcosms prior to receipt of this information in December 2004. The injections were suspended until groundwater could be obtained. Upon receipt of groundwater in January 2005, samples of this groundwater were submitted to the laboratory for perchlorate determination. The groundwater also exhibited low levels of perchlorate (approx. 20 ppb). Two gallons of the groundwater were spiked, using sodium perchlorate reagent, to 150,000 ppb perchlorate to use in spiking the soil microcosms.

One half of a liter (0.5 L) of the 150,000 ug/l perchlorate solution (75 mg perchlorate) was added to all five microcosms and thoroughly mixed. Samples were again taken to establish a baseline. Residual perchlorate concentrations in the control were 8800 ug/l and 2200-3400 μg/l in microcosms 002 through 004. Microcosm 005, however, showed non-detectable levels perchlorate after being spiked.

Microcosms 002 and 003 were treated for 90 minutes on Feb. 18, 2005 and Mar. 17, 2005. Microcosms 004 and 005 were treated for 90 minutes on Apr. 15, 2005 and Apr. 25, 2005. Microcosm 004 and 005 were sampled May 5, 2005 for perchlorate. Microcosms 002 and 003 were sampled on Apr. 15, 2005 for perchlorate.

Discussion of Microcosm #005 with DCPK

After receipt of the Feb. 18, 2005 sampling results, it was decided to respike microcosm #005 with 50% of the original perchlorate spike mass to determine if some error in sample handling had occurred. 250 ml of 150,000 mg/l perchlorate contaminated groundwater (37.5 mg perchlorate) was added to the microcosm on March 16, 2005, thoroughly mixed and resampled. Again the perchlorate concentration results were non-detectable.

It was decided that enzyme activity created prior to perchlorate addition may be affecting this microcosm due to the added DCPK factor. The microcosm was spiked again on Apr. 15, 2005 with one liter of 150000 μg/l perchlorate contaminated groundwater (150 mg perchlorate), the microcosm was mixed thoroughly, and then resampled. The results from this spike resulted in a residual of 6900 μg/l perchlorate as a baseline.

Two additional injections of gas phase carbon blend and DCPK in the base mixture, as described above were conducted and the #005 microcosm was sampled May 5, 2005.

Results of Chemical Analyses

Microcosms #003, #004 and #005 all demonstrated complete reduction of perchlorate to non-detectable levels. The control microcosm (#001) exhibited a 21.59% reduction in perchlorate; the microcosm that received the base treatment only (#002) experienced a 53.94% reduction in perchlorate. No perchlorate reduction byproducts (chlorate and chlorite) were detectable in any sample.

The results of the chemical analyses and reductions are presented in Table 2

TABLE 2

| | | | | | | | | | | | Nitrate/ |
|---|---|---|---|---|---|---|---|---|---|---|---|
| | | | | Perchlorate | Chlorate | Chlorite | Moisture | Fe 2+ | Cl- | SO4 | nitrite |
| | | | Date | ug/kg | ug/kg | ug/kg | % | mg/kg | mg/kg | mg/kg | mg/kg |
| Groundwater | | 1 of 5 | Jan. 05, 2005 | 20.7 | ND | ND | ND | ND | ND | ND | ND |
| (as received) | | | | | | | | | | | |
| Groundwater | | 5 of 5 | Jan. 05, 2005 | 20.0 | ND | ND | ND | ND | ND | ND | ND |
| (as received) | | | | | | | | | | | |
| Spiked | | 1 of 5 | May 05, 2005 | 150000 | <10 | <20 | 100 | ND | ND | ND | ND |
| Groundwater | | | | | | | | | | | |
| Soil Control | Bucket 001 | Control | Nov. 27, 2004 | <14.1 | ND | ND | 29 | <1 | <40 | <100 | <2 |
| Soil Control | Bucket 001 | Control | Feb. 18, 2005 | 8800 | <61 | <120 | 34 | ND | ND | ND | ND |
| Spiked | | | | | | | | | | | |
| Soil Control | Bucket 001 | Control | Apr. 15, 2005 | 6900 | <64 | <130 | 38 | ND | ND | ND | ND |
| Spiked | | | | | | | | | | | |
| | | | % Reduction | 21.59 | | | | | | | |
| The following | | | | | | | | | | | |
| Microcosms | | | | | | | | | | | |
| were all | | | | | | | | | | | |
| sparged with a | | | | | | | | | | | |
| carrier gas 96% | | | | | | | | | | | |
| Nitrogen and | | | | | | | | | | | |
| 1% hydrogen, | | | | | | | | | | | |
| and 3% | | | | | | | | | | | |
| propane | | | | | | | | | | | |
| As received | Bucket 002 | TEP/TMP only | Nov. 27, 2004 | <12.3 | ND | ND | 18.8 | <1 | <40 | <100 | <2 |
| Spike 1 (75 | Bucket 002 | TEP/TMP only | Feb. 18, 2005 | 3400 | <49 | <98 | 18 | ND | ND | ND | ND |
| mg perchlorate) | | | | | | | | | | | |
| Post treatment | Bucket 002 | TEP/TMP only | Apr. 15, 2005 | 1600 | <49 | <98 | 19 | ND | ND | ND | ND |
| | | | % Reduction | 52.94 | | | | | | | |
| As received | Bucket 003 | TEP/TMP + | Nov. 27, 2004 | <12.3 | ND | ND | 18.9 | <1 | <40 | <100 | <2 |
| | | acetate | | | | | | | | | |
| Spike 1 (75 | Bucket 003 | TEP/TMP + | Feb. 18, 2005 | 3300 | <49 | <99 | 19 | ND | ND | ND | ND |
| mg perchlorate) | | acetate | | | | | | | | | |
| Post treatment | Bucket 003 | TEP/TMP + | Apr. 15, 2005 | <4.9 | <25 | <50 | 19 | ND | ND | ND | ND |
| | | acetate | % Reduction | >99.85 | | | | | | | |
| As received | Bucket 004 | TEP/TMP + | Nov. 27, 2004 | <12.4 | ND | ND | 19.2 | <1 | <40 | <100 | <2 |
| | | ethanol | | | | | | | | | |
| Spike 1 (75 | Bucket 004 | TEP/TMP + | Feb. 18, 2005 | 2200 | 59 | <100 | 20 | ND | ND | ND | ND |
| mg perchlorate) | | ethanol | | | | | | | | | |
| Post treatment | Bucket 004 | TEP/TMP + | May 05, 2005 | <5.2 | <100 | <52 | 23 | 2765 | <250 | 3536 | <1 |
| | | ethanol | % Reduction | >99.76 | | | | | | | |
| As received | Bucket 005 | DCPK Blend | Nov. 27, 2004 | <13.3 | ND | ND | 24.6 | <1 | <40 | <100 | <2 |
| Spike 1 (75 | Bucket 005 | DCPK Blend | Feb. 18, 2005 | <5.3 | <50 | <100 | 25 | ND | ND | ND | ND |
| mg perchlorate) | | | | | | | | | | | |
| Spike 2 (37.5 | Bucket 005 | DCPK Blend | Mar. 16, 2005 | <5.5 | <110 | <55 | 28 | ND | <250 | 1129 | <1 |
| mg perchlorate) | | | | | | | | | | | |
| Spike 3 (150 | Bucket 005 | DCPK Blend | Apr. 15, 2005 | 6900 | <130 | <64 | 38 | ND | ND | ND | ND |
| mg perchlorate) | | | | | | | | | | | |
| Post treatment | Bucket 005 | DCPK Blend | May 05, 2005 | <5.5 | <110 | <55 | 28 | 2227 | <250 | 1176 | <1 |
| | | | % Reduction | >99.92 | | | | | | | |

ND = No Data Collected
Bucket 002, 003, 004 and 005 all received propane and hydrogen in nitrogen gas carrier
Soil Control received no amendment
Groundwater concentrations are ug/l

Perchlorate Reducing Microbe Counts

Perchlorate reducing microbes (PRMs) were present and enumerated by Most Probable Number (MPN) counts in all samples pre and post-treatment. The addition of gas phase acetate was found to increase the PRM populations in a microcosm, approaching three orders of magnitude.

The addition of gas phase ethanol was found to increase the PRM populations in a microcosm approaching one order of magnitude.

The addition of DCPK with a gas-phase carbon blend was found to increase the PRM population over three orders of magnitude; this combination produced the greatest positive change in perchlorate reducing microbial population and the greatest absolute numbers of PRM. Higher numbers of microorganisms were cultured in the microcosm that was fed the DCPK blend. Over an order of magnitude higher population levels (7.49×10⁷ MPN) were observed in this microcosm.

The results of the perchlorate reducing microbe counts (MPN) are presented in Table 3.

TABLE 3

| | | | | Most Probabable Number | Chlorite Dismutase Gene |
|---|---|---|---|---|---|
| Description | Sample # | Added Gas Phase Blend | Date | Chlorate Reducers | cld |
| Soil Control | Bucket 001 | Control | Dec. 07, 2004 | 9.33 +− 7.27 E5 | Positive |
| Soil Control Post-treatment | Bucket 001 | Control | May 12, 2005 | 9.33 +− 4.17 E5 | Negative |
| | | | % Reduction | 21.59 | |
| The following Microcosms | | | | | |
| were all sparged with a | | | | | |
| carrier gas 96% Nitrogen | | | | | |
| and 1% hydrogen, and 3% | | | | | |
| propane | | | | | |
| As received | Bucket 002 | TEP/TMP only | Dec. 07, 2004 | 4.27 +− 3.24 E4 | Positive |
| Post treatment | Bucket 002 | TEP/TMP only | May 12, 2005 | 2.31 +− 1.33 E5 | Negative |
| | | | % Reduction | 52.94 | |
| As received | Bucket 003 | TEP/TMP + acetate | Dec. 07, 2004 | 4.27 +− 3.24 E3 | Positive |
| Post treatment | Bucket 003 | TEP/TMP + acetate | May 12, 2005 | 2.31 +− 1.33 E6 | Positive |
| | | | % Reduction | >99.85 | |
| As received | Bucket 004 | TEP/TMP + ethanol | Dec. 07, 2004 | 2.4 +− 1.92 E5 | Positive |
| Post treatment | Bucket 004 | TEP/TMP + ethanol | May 12, 2005 | 2.31 +− 1.33 E6 | Positive |
| | | | % Reduction | >99.76 | |
| As received | Bucket 005 | DCPK Blend | Dec. 07, 2004 | 2.40 +− 1.92 E4 | Positive |
| Post treatment | Bucket 005 | DCPK Blend | May 12, 2005 | 7.49 +− 3.35 E7 | Positive |
| | | | % Reduction | >99.92 | |

Bucket 002, 003, 004 and 005 all received propane and hydrogen in nitrogen gas carrier
Soil Control received no amendment

Chlorite Dismutase Gene cld Probe Results

The chlorite dismutase gene (cld) was positively identified in all samples as received. The chlorite dismutase gene was not found or was obscured in the Control (#001) and base fed only (#002) microcosms at the conclusion of the test. The chlorite dismutase gene was found in microcosm #003, #004 (very faint) and #005 at the test conclusion. The results of the chlorite dismutase gene identification are presented in Table 3 above.

Field Parameter Analytical Results

At the time of sample collection as decribed above, “field analysis” of water samples were performed that included an evaluation of dissolved oxygen, pH, specific conductivity, and oxidation/reduction potential (ORP) for each sample.

### CONCLUSIONS

Ethyl acetate and ethanol, individually, and a blend of gas phase carbon sources with DCPK, can be introduced in a gas phase to the perchlorate contaminated microcosms.

Each of the gas blends provided a sufficient microbial environment to promote complete degradation of perchlorate in the microcosms.

The addition of gas phase acetate was found to increase the PRM populations in a microcosm, approaching three orders of magnitude.

The addition of gas phase ethanol was found to increase the PRM populations in a microcosm approaching one order of magnitude.

The addition of DCPK with a gas-phase carbon blend was found to increase the PRM population over three orders of magnitude; this combination produced the greatest positive change in perchlorate reducing microbial population and the greatest absolute numbers of PRM.

350% more perchlorate was degraded by the microcosm that had added DCPK, as compared to the ethyl acetate or ethanol only fed microcosms. Since the reduction of perchlorate occurred immediately after a perchlorate spike occurred, in two separate instances, this is hypothesized to be the result of increased enzyme production by the microcosm.

Higher numbers of microorganisms were cultured in the microcosm that was fed the DCPK blend. Over an order of magnitude higher population levels (7.49×10⁷ MPN) were observed in this microcosm.

The inventors believe that DCPK can result in the preferred proliferation of perchlorate reducing microorganisms, expressing certain genes, and containing membrane bound heme enzymes beneficial to the perchlorate reducing process.

It will be apparent to those who are skilled in the art that numerous changes and substitutions can be made to the embodiments described above without departing from the spirit and scope of the present invention. Any and all publicly available documents cited herein are specifically incorporated herein in their entireties.

What is claimed is:

1. A gaseous composition comprising (a) at least one gaseous microbial metabolic inducer and (b) a carrier gas.

2. The gaseous composition of claim 1, wherein said inducer is selected from the group consisting of dicyclopropylketone, dicyclopropyl methanol, ethylacetate, diethylether, isopropyl-β-thiogalactopyronoside, and combinations thereof.

3. The gaseous composition of claim 2, wherein said inducer is dicyclopropylketone.

4. The gaseous composition of claim 3, wherein said dicyclopropylketone is present in a concentration from about 0.0005% to about 10% volume to volume.

5. The gaseous composition of claim 4, wherein said concentration is about 0.05% volume to volume.

6. The gaseous composition of claim 1, wherein said carrier gas is selected from the group consisting of air, an inert gas, and combinations thereof.

7. The gaseous composition of claim 1, further comprising hydrogen (H₂).

8. The gaseous composition of claim 1, further comprising at least one gas phase nutrient, said gas phase nutrient selected from the group consisting of triethylphosphate, trimethylphosphate, tributylphosphate, nitrous oxide, and combination thereof.

9. The gaseous composition of claim 1, further comprising a gas phase carbon source selected from the group consisting of a volatile alkanes, volatile alcohols, volatile esters, and combinations thereof.

10. The gaseous composition of claim 1, further comprising steam or humidified air.

11. The gaseous composition of claim 1, wherein said inducer is dicyclopropylketone and said carrier gas is nitrogen (N₂), and wherein said composition further comprises (c) triethylphosphate, (d) hydrogen (H₂), (e) propane, (f) optionally, ethanol, and, (g) optionally, ethylacetate.

12. The gaseous composition of claim 11, wherein the composition comprises (f) ethanol and (g) ethylacetate,

13. A method of stimulating microbial degradation of at least one pollutant in a subsurface environment, said method comprising contacting the subsurface environment with (a) a gaseous composition comprising at least one gaseous microbial metabolic inducer and (b) a carrier gas.

14. The method of claim 13, wherein said inducer is selected from the group consisting of dicyclopropylketone, dicyclopropyl methanol, ethylacetate, diethylether, isopropyl-β-thiogalactopyronoside, and combinations thereof.

15. The method of claim 14, wherein said inducer is dicyclopropylketone.

16. The method of claim 15, wherein said dicyclopropylketone is present in a concentration from about 0.0005% to about 10% volume to volume.

17. The method of claim 16, wherein said concentration is about 0.05% volume to volume.

18. The method of claim 13, wherein said carrier gas is selected from the group consisting of air, an inert gas, and combinations thereof.

19. The method of claim 13, wherein said gaseous composition further comprises hydrogen (H₂).

20. The method of claim 13, wherein said gaseous composition further comprises a phase microbial nutrient selected from the group consisting of triethylphosphate, trimethylphosphate, tributylphosphate, nitrous oxide. and combinations thereof.

21. The method of claim 13, wherein said gaseous composition further comprises a gas phase carbon source selected from the group consisting of volatile alkanes, volatile alcohols, volatile esters, and combinations thereof.

22. The method of claim 13, wherein said gaseous composition further comprises team or humidified air.

23. The method of claim 13, wherein said subsurface environment comprises subsoil, groundwater, or both.

24. The method of claim 13, wherein said contacting is performed continuously or in pulses.

25. The method of claim 13, wherein said pollutant is selected from the group consisting of alkanes, alkenes, chlorinated alkanes, chlorinated alkenes, aromatic compounds, cyclotetramethylenetetranitramine (HMX), cyclotrimethylenetrinitramine (RDX), trinitrotoluene (TNT), perchlorate and combinations thereof.

26. The method of claim 13, wherein said pollutant comprises perchlorate and said gaseous composition comprises
(a) dicyclopropylketone as said gaseous inducer,
(b) nitrogen as said carrier gas,
(c) a gas phase phosphate nutrient comprising triethylphosphate, trimethylphosphate, or a combination thereof;
(d) hydrogen (H₂),
(e) propane,
(f) optionally, ethanol; and,
(g) optionally, ethylacetate.

27. The method of claim 26, wherein said composition comprises ethanol and ethylacetate.
